(19) United States
(12) Patent Application Publication
(10) Pub. No.: US 2023/0277105 A1
(43) Pub. Date: Sep. 7, 2023

(54) Blood Access Device with Integrated Blood Diagnostics

(71) Applicant: Becton, Dickinson and Company, Franklin Lakes, NJ (US)

(72) Inventors: Jonathan Karl Burkholz, Salt Lake City, UT (US); Megan S. Scherich, Salt Lake City, UT (US); John M. Lackey, West Valley City, UT (US)

(21) Appl. No.: 18/117,765

(22) Filed: Mar. 6, 2023

Related U.S. Application Data
(60) Provisional application No. 63/317,304, filed on Mar. 7, 2022.

(51) Int. Cl. A61B 5/15

(52) U.S. Cl. CPC A61B 5/150992; A61B 5/15003

(57) ABSTRACT

A blood sample collection system includes a blood access device having a fluid flow path and a diagnostic device configured to receive a sample of blood, where the diagnostic device is in fluid communication with the fluid flow path, and where the diagnostic device is configured to be detached from the blood access device.

### CROSS-REFERENCE TO RELATED APPLICATION

The present application claims priority to U.S. Provisional Application No. 63/317,304, entitled “Blood Access Device with Integrated Blood Diagnostics” filed Mar. 7, 2022, the entire disclosure of which is hereby incorporated by reference in its entirety.

### BACKGROUND OF THE INVENTION

### Field of the Invention

The present disclosure relates to a blood access device with integrated blood diagnostics.

### Description of Related Art

Catheters are frequently utilized to administer fluids into and out of the body. Patients in a variety of settings, including in hospitals and in home care, receive fluids, pharmaceuticals, and blood products via a vascular access device inserted into a patient's vascular system. Catheters of various types and sizes have been used extensively in a variety of procedures including, but not limited to, treating an infection, providing anesthesia or analgesia, providing nutritional support, treating cancerous growths, maintaining blood pressure and heart rhythm, and many other clinical uses. A common vascular access device is a plastic catheter that is inserted into a patient's vein. The catheter length may vary from a few centimeters for peripheral access to many centimeters for central access. The catheter is commonly incorporated into a catheter adapter to aid in the ease of use, accessibility, and utility of the catheter. A catheter adapter may be adapted to house one end of the catheter such that one end of the catheter is supported by the catheter adapter and the body and tip of the catheter extends beyond a first end of the catheter adapter. A catheter adapter generally further includes a second end adapted to receive additional infusion components for use with the catheter. For example, the second end of a catheter adapter may include a set of threads for attaching an intravenous line or for coupling a syringe to the catheter adapter thereby providing access to the patient's vasculature via the attached catheter.

The catheter may be inserted transcutaneously. When inserted transcutaneously, the insertion of the catheter is commonly aided by an introducer needle. The introducer needle is commonly housed inside the lumen of the catheter such that the gauge of the needle approximates the inner diameter of the catheter. The needle is positioned within the catheter such that the needle tip extends beyond the tip of the catheter whereby the needle is used to penetrate the patient's vein and provide an opening for insertion of the catheter.

In order to verify proper placement of the introducer needle and/or the catheter in the blood vessel, a clinician generally confirms that there is “flashback” of blood in a flashback chamber of the catheter assembly. Once placement of the needle has been confirmed, the clinician may temporarily occlude flow in the vasculature and remove the needle, leaving the catheter in place for future blood withdrawal, fluid infusion, or probe access.

Recent developments in the peripheral intravenous catheter (PIVC) field have led to the emergence of technologies designed to facilitate blood draw and an in-dwelling PIVC. These devices have focused on the ability to reliably collect a high-quality blood sample and reduce hemolysis. The main method by which these devices work is by inserting a guide wire, probe, tube, or other instrument through the lumen of the catheter. This arrangement creates a fluid path through any thrombus or fibrin that may be occluding the tip of the catheter. A syringe or vacutainer may then be used to collect blood samples without needing to subject the patient to additional needle sticks.

Point-of-care (POC) testing includes the use of a portable testing system that can quickly deliver results using small volume blood samples, such as for glucose testing. Blood samples are typically collected via finger pricks or through existing vascular access. However, the majority of vascular access devices are not optimized for the collection of small blood samples. The current field is moving toward personalized healthcare with the goal of POC testing that can provide rapid diagnostic results with a smaller volume of blood. There is a current need to provide POC blood sample collection options from a PIVC in anticipation for increased POC diagnostic testing.

The subject matter claimed herein is not limited to embodiments that solve any disadvantages or that operate only in environments such as those described above. Rather, this background is only provided to illustrate one example technology area where some implementations described herein may be practiced.

### SUMMARY OF THE INVENTION

In one aspect or embodiment, a blood sample collection system includes a blood access device having a fluid flow path and a diagnostic device configured to receive a sample of blood, where the diagnostic device is in fluid communication with the fluid flow path, and where the diagnostic device is configured to be detached from the blood access device.

The blood access device may include a housing, an instrument disposed within the housing, and an advancement element, where in response to movement of the advancement element with respect to the housing, the instrument is configured to advance beyond a distal end of the housing.

The diagnostic device may be configured to receive a sample volume of 0.3 to 500 microliters. The diagnostic device may be configured to receive a sample volume of 0.3 to 100 microliters. The blood access device may be a peripheral intravenous catheter. The blood access device may include extension tubing. The diagnostic device may be a diagnostic test cartridge.

The blood access device may include one of a cannula and septum, with the diagnostic test cartridge including the other of the cannula and the septum, and the cannula configured to extend through the septum to place the fluid flow path of the blood access device in fluid communication with the diagnostic test cartridge. A body of the diagnostic test cartridge may be cylindrical. A body of the diagnostic test cartridge may be planar.

The advancement element may include a cannula and the diagnostic device may include a septum, with the cannula configured to extend through the septum to place the fluid flow path of the blood access device in fluid communication with the diagnostic device. The housing of the blood access device may include a distal opening configured to receive at least a portion of the diagnostic device. The instrument may include a flow tube or a helical coil. The diagnostic device may be a diagnostic test strip, a lateral flow assay, or a visual indicator.

In a further aspect or embodiment, a blood sample collection system includes a medical connector having a fluid flow path and a diagnostic device configured to receive a sample of blood, where the diagnostic device is in fluid communication with the fluid flow path, and where the diagnostic device is configured to be detached from the medical connector.

The medical connector may include one of a male luer connector and female luer connector, with the diagnostic device including the other of the male luer connector and the female luer connector. The medical connector may be a needle-free medical connector. The diagnostic device may be a diagnostic test cartridge.

### BRIEF DESCRIPTION OF THE DRAWINGS

The above-mentioned and other features and advantages of this disclosure, and the manner of attaining them, will become more apparent, and the disclosure itself will be better understood by reference to the following descriptions of embodiments of the disclosure taken in conjunction with the accompanying drawings, wherein:

FIG. 1 is a top view of a blood access device and diagnostic cartridge according to one aspect or embodiment of the present application;

FIG. 2 is a top view of the blood access device and diagnostic cartridge of FIG. 1, showing a post-advancement position of the blood access device;

FIG. 3 is a top view of the blood access device and diagnostic cartridge of FIG. 1, showing a post-advancement position of the blood access device and blood within a diagnostic cartridge;

FIG. 4 is a top view of the blood access device and diagnostic cartridge of FIG. 1, showing a retracted position of the blood access device and removal of a diagnostic cartridge;

FIG. 5 is a front view of a test device according to one aspect or embodiment of the present application, showing a diagnostic cartridge being inserted into the test device;

FIG. 6A is a front view of the blood access device and diagnostic cartridge of FIG. 1, showing a post-advancement position without a diagnostic cartridge;

FIG. 6B is a front view of the blood access device of FIG. 1, showing a post-advancement position with a diagnostic cartridge;

FIG. 6C is a front view of the blood access device of FIG. 1, showing a post-advancement position with blood received within a diagnostic cartridge;

FIG. 6D is a front view of the blood access device of FIG. 1, showing a post-advancement position with a diagnostic cartridge being removed;

FIG. 6E is a front view of the blood access device of FIG. 1, showing a post-advancement position with a further diagnostic cartridge being attached;

FIG. 7 is a front view of a blood access device and diagnostic cartridge according to one aspect or embodiment of the present application;

FIG. 8 is a front view of a blood access device and diagnostic cartridge according to a further aspect or embodiment of the present application;

FIG. 9 is a perspective view of a blood access device and diagnostic cartridge according to a further aspect or embodiment of the present application;

FIG. 10 is a cross-sectional view of the blood access device and diagnostic cartridge of FIG. 9;

FIG. 11 is a perspective view of the blood access device and diagnostic cartridge of FIG. 9, showing blood within the diagnostic cartridge;

FIG. 12A is a top view of a blood access device and diagnostic test strip according to one aspect or embodiment of the present application;

FIG. 12B is a top view of the blood access device and diagnostic test strip of FIG. 12A, showing a post-advancement position of the blood access device;

FIG. 12C is a top view of the blood access device and diagnostic test strip of FIG. 12A, showing a retracted position of the blood access device and removal of the diagnostic test strip;

FIG. 13A is a top view of a blood access device and lateral flow assay according to one aspect or embodiment of the present application;

FIG. 13B is a top view of the blood access device and lateral flow assay of FIG. 13A, showing a post-advancement position of the blood access device and with blood present in the lateral flow assay;

FIG. 14A is a top view of a blood access device and diagnostic test according to one aspect or embodiment of the present application, showing a post-advancement position of the blood access device;

FIG. 14B is a top view of the blood access device and diagnostic test of FIG. 14A, showing a retracted position of the blood access device and with blood present in the diagnostic test;

FIG. 15A is a perspective view of the diagnostic test of FIG. 14A;

FIG. 15B is a schematic view of a diagnostic test results key according to one aspect or embodiment of the present application;

FIG. 16 is a top view of a blood access device and diagnostic cartridge according to one aspect or embodiment of the present application, showing a large sample collection fluid path;

FIG. 17A is a perspective view of a medical connector and diagnostic cartridge according to one aspect or embodiment of the present application;

FIG. 17B is a perspective view of a medical connector and diagnostic cartridge according to one aspect or embodiment of the present application;

FIG. 18 is a schematic view of a syringe-based blood draw device and diagnostic device according to one aspect or embodiment of the present application;

FIG. 19 is a front view of a blood access device and diagnostic device according to one aspect or embodiment of the present application;

FIG. 20 is a front view of a blood access device and diagnostic test strip according to one aspect or embodiment of the present application;

FIG. 21 is a perspective view of a blood access device and diagnostic device according to one aspect or embodiment of the present application;

FIG. 22 is a schematic view of a blood access device and diagnostic device according to one aspect or embodiment of the present application; and

FIG. 23 is a perspective view of a medical connector and diagnostic cartridge according to one aspect or embodiment of the present application.

Corresponding reference characters indicate corresponding parts throughout the several views. The exemplifications set out herein illustrate exemplary embodiments of the disclosure, and such exemplifications are not to be construed as limiting the scope of the disclosure in any manner.

### DETAILED DESCRIPTION OF THE INVENTION

Spatial or directional terms, such as “left”, “right”, “inner”, “outer”, “above”, “below”, and the like, are not to be considered as limiting as the invention can assume various alternative orientations.

For purposes of the description hereinafter, the terms “upper”, “lower”, “right”, “left”, “vertical”, “horizontal”, “top”, “bottom”, “lateral”, “longitudinal”, and derivatives thereof shall relate to the invention as it is oriented in the drawing figures. However, it is to be understood that the invention may assume various alternative variations, except where expressly specified to the contrary. It is also to be understood that the specific devices illustrated in the attached drawings, and described in the following specification, are simply exemplary aspects of the invention.

Unless otherwise indicated, all ranges or ratios disclosed herein are to be understood to encompass the beginning and ending values and any and all subranges or subratios subsumed therein. For example, a stated range or ratio of “1 to 10” should be considered to include any and all subranges or subratios between (and inclusive of) the minimum value of 1 and the maximum value of 10; that is, all subranges or subratios beginning with a minimum value of 1 or more and ending with a maximum value of 10 or less.

The terms “first”, “second”, and the like are not intended to refer to any particular order or chronology, but refer to different conditions, properties, or elements.

As used herein, “at least one of” is synonymous with “one or more of”. For example, the phrase “at least one of A, B, and C” means any one of A, B, or C, or any combination of any two or more of A, B, or C. For example, “at least one of A, B, and C” includes one or more of A alone; or one or more of B alone; or one or more of C alone; or one or more of A and one or more of B; or one or more of A and one or more of C; or one or more of B and one or more of C; or one or more of all of A, B, and C.

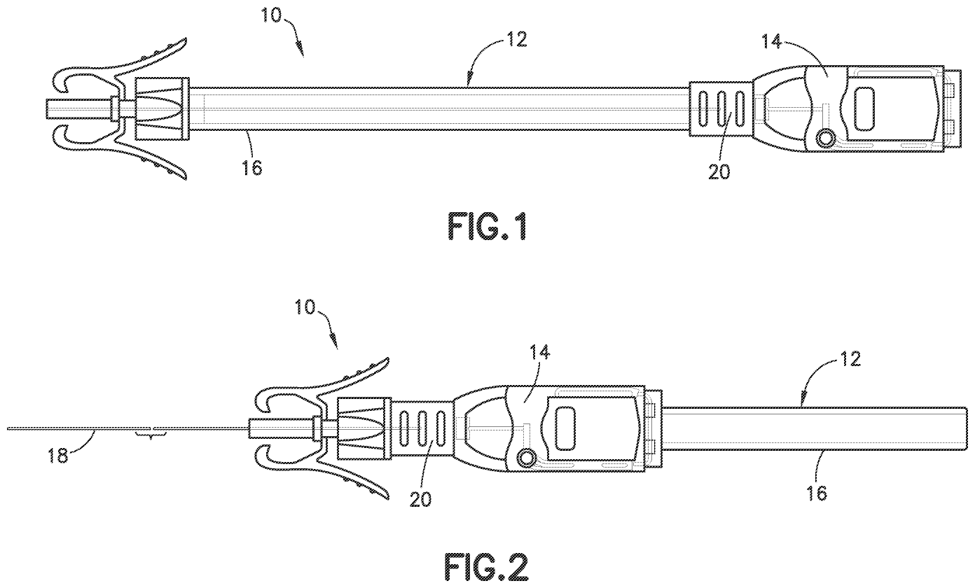

Referring to FIGS. 1-6E, in one aspect or embodiment of the present application, a blood sample collection system 10 includes a blood access device 12 having a fluid flow path and a diagnostic device 14 configured to receive a sample of blood. The diagnostic device 14 is in fluid communication with the fluid flow path and is configured to be detached from the blood access device 12.

In one aspect or embodiment, the blood access device 12 includes a housing 16, an instrument 18 disposed within the housing 16, and an advancement element 20. In response to movement of the advancement element 20 with respect to the housing 16, the instrument 18 is configured to advance beyond a distal end of the housing 16. The instrument 18 may be a flow tube, although other suitable arrangements may be utilized. The blood access device 12 may be configured to be connected to a peripheral intravenous catheter, a peripherally-inserted central catheter, or a midline catheter. In one aspect or embodiment, the blood access device 12 is coupled to an indwelling catheter via a needleless connector (not shown). In some aspects or embodiments, the blood access device 12 may be the PIVO™ blood draw device commercially available from Velano Vascular. In one embodiment, the blood access device 12 is the same or similar to the blood draw device shown in U.S. Pat. No. 11,090,461, which is hereby incorporated by reference in its entirety.

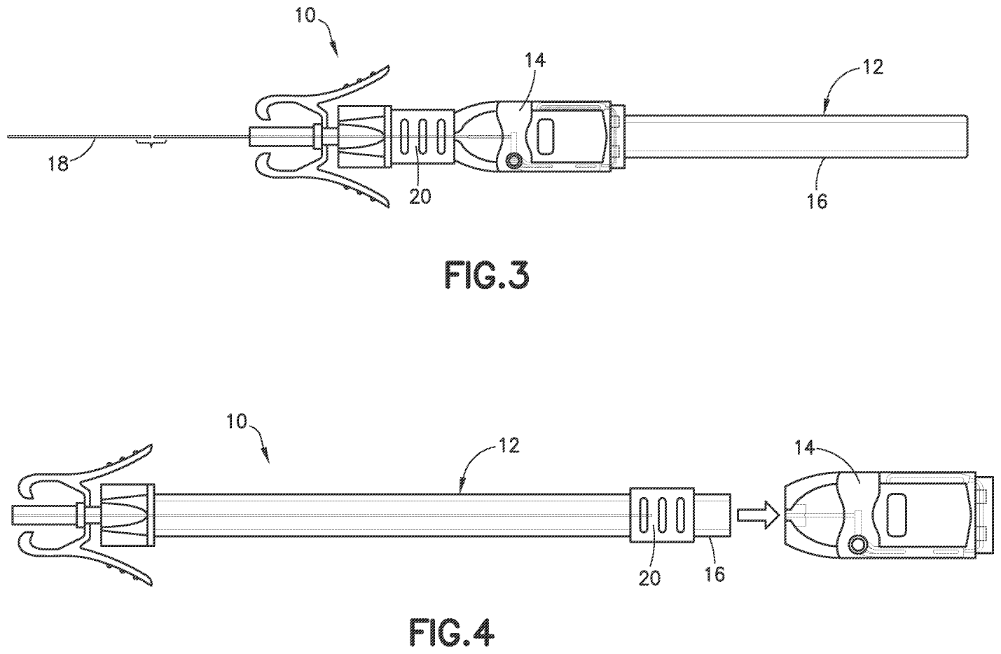

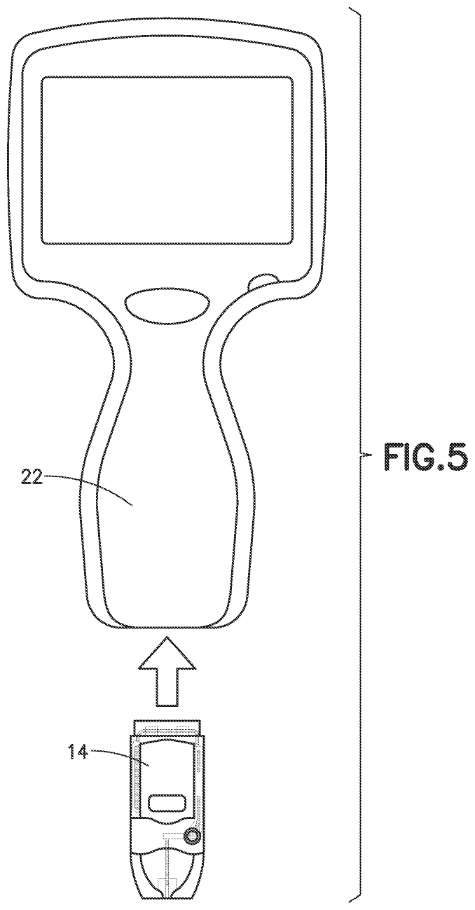

Referring to FIGS. 1-5, during use, the blood sample collection system 10 is connected to an indwelling catheter with the blood access device 12 initially in the pre-advancement or retracted position (FIG. 1). The instrument 18 of the blood access device 12 is moved or advanced to the post-advancement position (FIG. 2), with the instrument 18 entering a patient's vasculature. Direct blood delivery to the diagnostic device 14 occurs (FIG. 3) with the direct transfer of blood from the patient's vasculature to the diagnostic device 14 via venous pressure and venting in the diagnostic device 14 or the fluid flow path of the blood access device 12. Capillary action may provide additional blood sample movement within a micro-fluidic pathway of the diagnostic device 14. In some aspects or embodiments, the instrument 18 of the blood access device 12 is moved to a retracted position and the diagnostic device 14 is removed (FIG. 4). The diagnostic device 14 can then be placed in a diagnostic test instrument 22 (FIG. 5). In some aspects or embodiments, the diagnostic device 14 is a diagnostic test cartridge, which may be the same or similar to an i-STAT CHEM 8+ diagnostic test cartridge commercially available from Abbott. In some aspects or embodiments, the diagnostic test instrument 22 may be the same or similar to an i-STAT Alinity diagnostic testing instrument commercially available from Abbott.

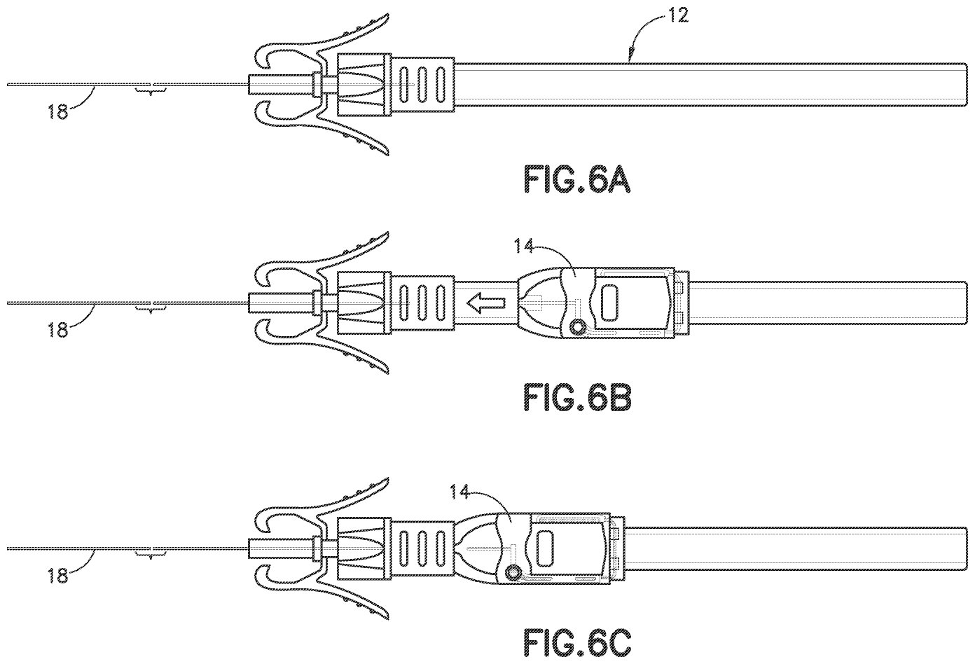

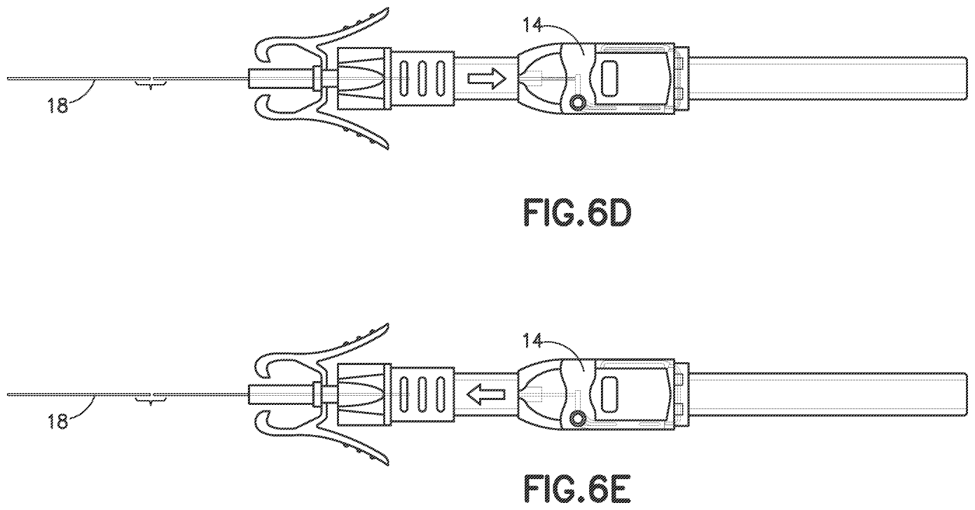

Referring to FIGS. 6A-6E, the blood access device 12 is configured to be advanced without the diagnostic device 14 (FIG. 6A) or with the diagnostic device 14 (FIG. 6B). After the diagnostic device 14 receives a blood sample (FIG. 6C), the diagnostic device 14 can be removed while the instrument 18 remains in the advanced position (FIG. 6D). An additional diagnostic device 14 can then be attached to the blood access device 12 (FIG. 6E) to obtain a second or additional blood sample.

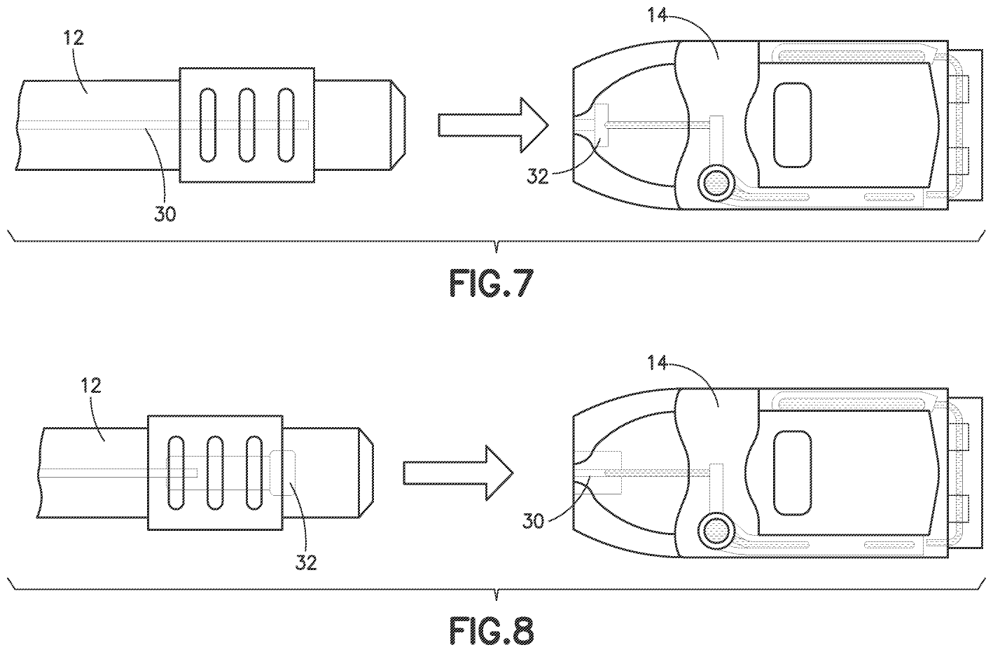

Referring to FIGS. 7 and 8, in one aspect or embodiment, the blood access device 12 includes one of a cannula 30 and septum 32, and the diagnostic device 14 includes the other of the cannula 30 and the septum 32, with the cannula 30 configured to extend through the septum 32 to place the fluid flow path of the blood access device 12 in fluid communication with the diagnostic device 14. In FIG. 7, the diagnostic device 14 includes the septum 32 and the blood access device 12 includes the cannula 30. In FIG. 8, the diagnostic device 14 includes the cannula 30 and the blood access device 12 includes the septum 32.

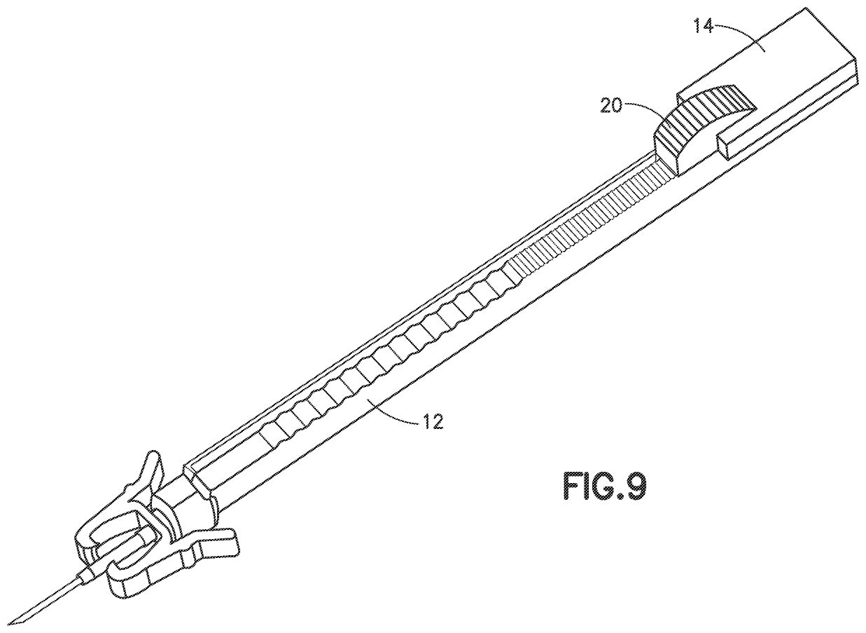

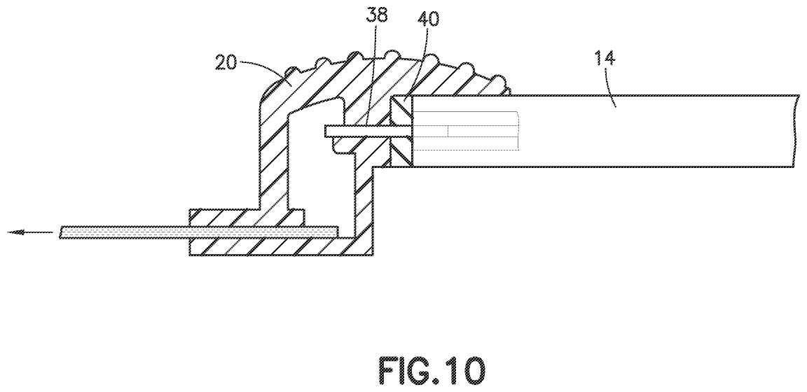

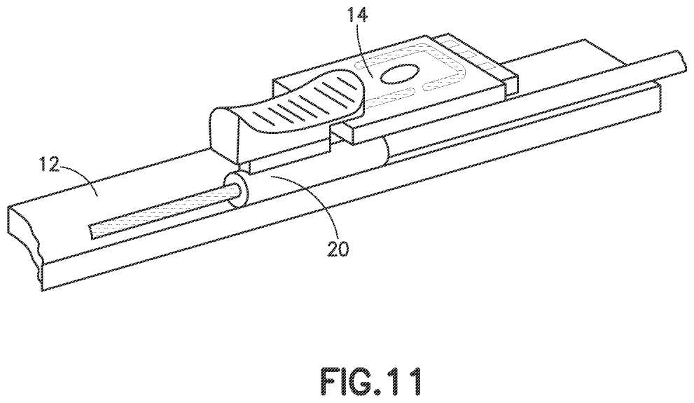

Referring to FIGS. 9-11, in one aspect or embodiment, the advancement element 20 of the blood access device 12 includes a cannula 38 and the diagnostic device 14 includes a septum 40, with the cannula 38 configured to extend through the septum 32 to place the fluid flow path of the blood access device 12 in fluid communication with the diagnostic device 14. The advancement element 20 may include a connector or other features to receive and hold the diagnostic device.

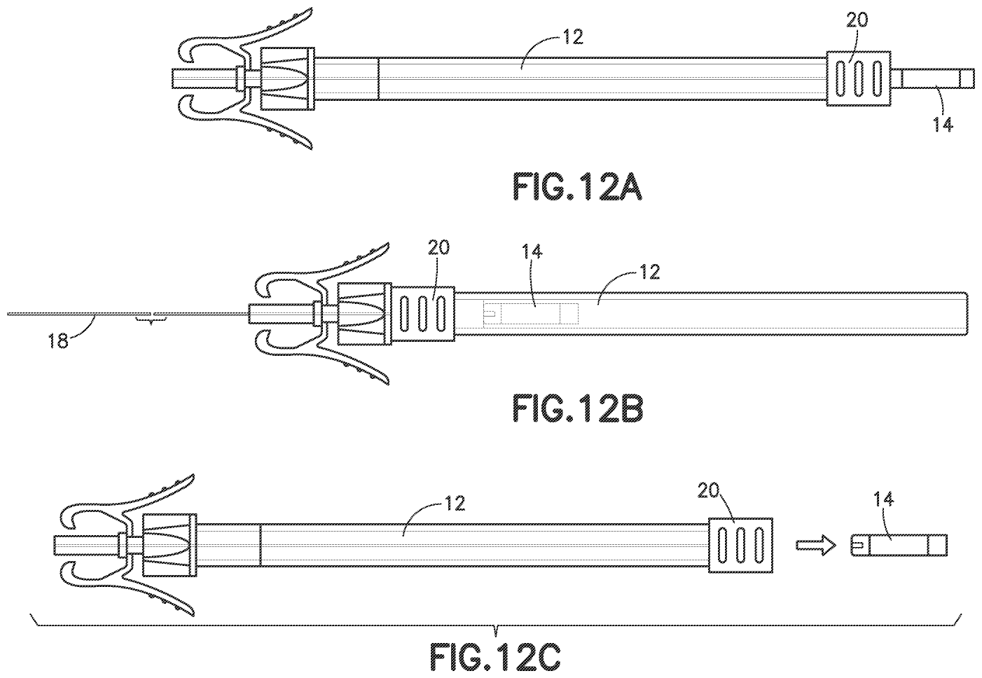

Referring to FIGS. 12A-12C, in one aspect or embodiment, the diagnostic device 14 is a diagnostic test strip. The diagnostic test strip may be advanced with the advancement element 20 (FIG. 12A), receive a blood sample (FIG. 12B), and then be removed for a diagnostic test (FIG. 12C).

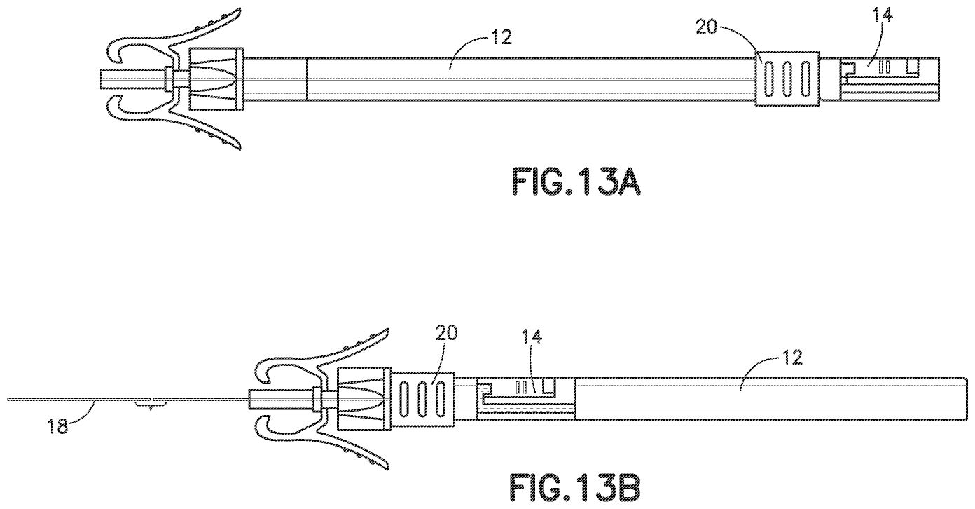

Referring to FIGS. 13A and 13B, in one aspect or embodiment, the diagnostic device 14 is a lateral flow assay. The lateral flow assay may be advanced with the advancement element 20 and receive a blood sample, which may be indicated via a blood visualization flashback chamber (FIG. 13B).

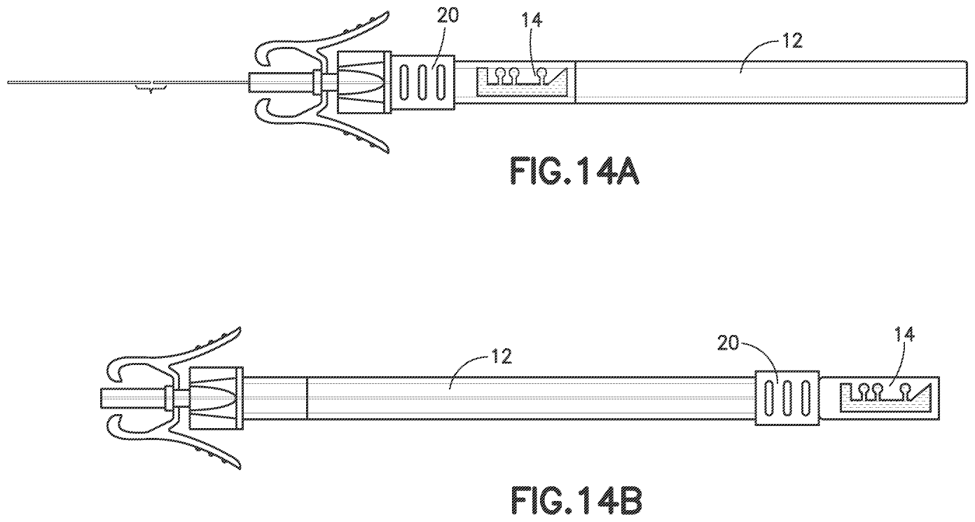

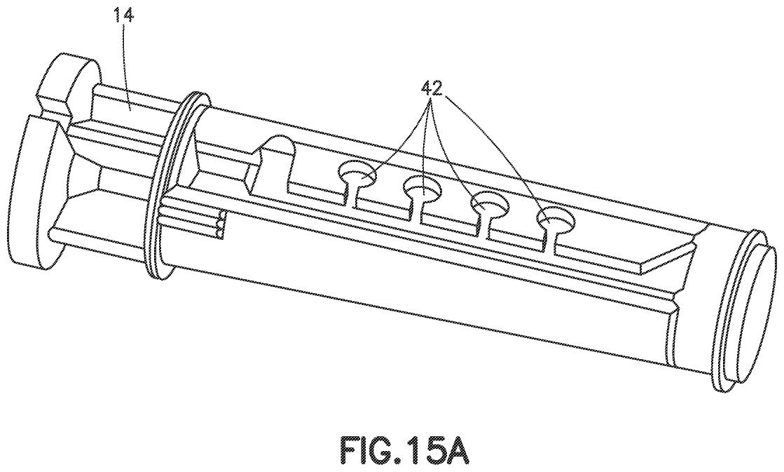

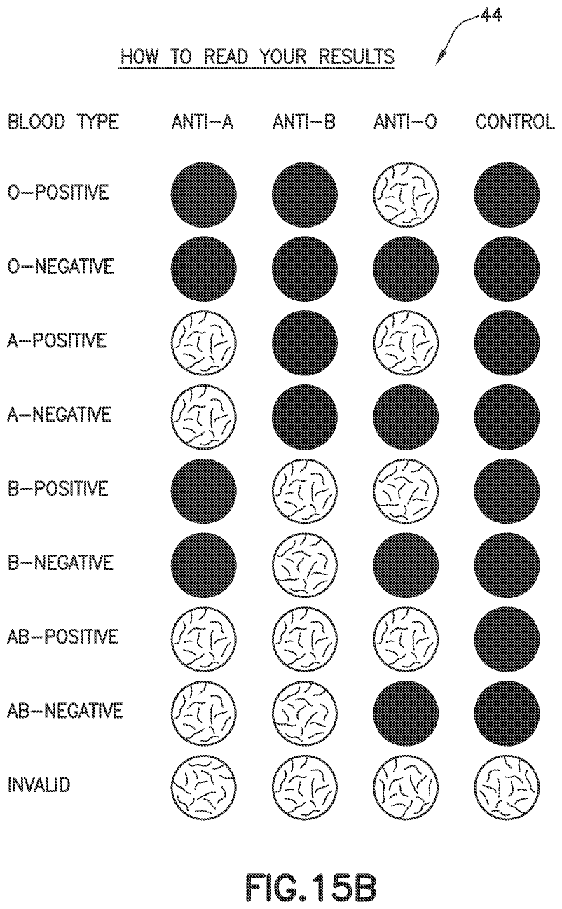

Referring to FIGS. 14A-15B, in one aspect or embodiment, the diagnostic device 14 is a visual diagnostic indicator. The visual diagnostic indicator may be advanced with the advancement element 20 and receive a blood sample (FIG. 14A). The visual diagnostic indicator may remain attached to the blood access device 12 or may be removable from the blood access device 12. The visual diagnostic indicator includes a plurality of indicators 42 (FIG. 15A), which can be compared to a diagnostic test key 44 (FIG. 15B) to determine the results of the test.

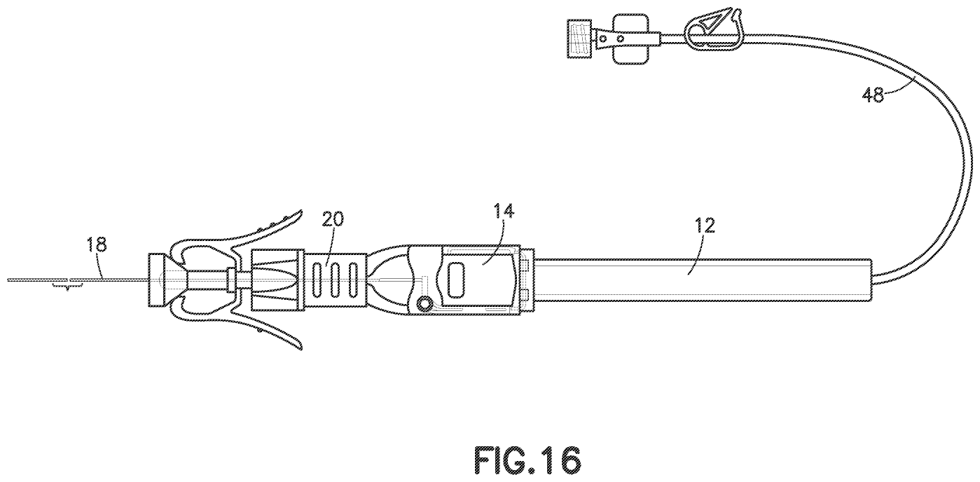

Referring to FIG. 16, in one aspect or embodiment, the blood access device 12 includes extension tubing 48 configured to receive larger volume blood samples. The extension tubing 48 may include a tube clamp and/or various medical connectors.

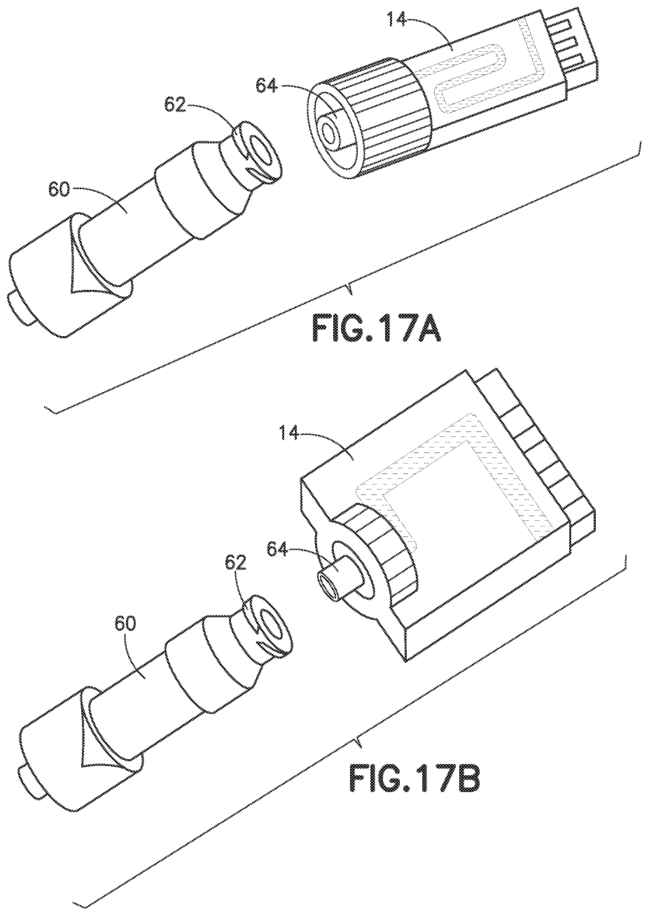

Referring to FIGS. 17A and 17B, in a further aspect or embodiment, the diagnostic device 14 is connected to a medical connector 60 having a fluid flow path. The medical connector 60 may be directly connected to a catheter, access port of an indwell catheter or extension set, venipuncture blood collection device, or connected to the blood access device via the extension tubing. In some aspects or embodiments, the medical connector 60 includes fluid path geometry configured to prevent hemolysis of the blood sample. In one aspect or embodiment, the fluid path geometry lowers a flow rate of blood within the fluid pathway, which may, in turn, lower a shear rate for hemolysis management. In some aspects or embodiments, the medical connector 60 includes one of the flow path geometries shown and described in U.S. Patent Application Publication No. 2021/10186394, which is hereby incorporated by reference in its entirety. The diagnostic device 14 may be cylindrical (FIG. 17A) or may be planar (FIG. 17B), although other suitable shapes and configurations may be utilized. The medical connector 60 may include a female luer connector 62, and the diagnostic device 14 may include a male luer connector 64 configured to connect to the female luer connector 62. The medical connector 60 may be a needle-free medical connector.

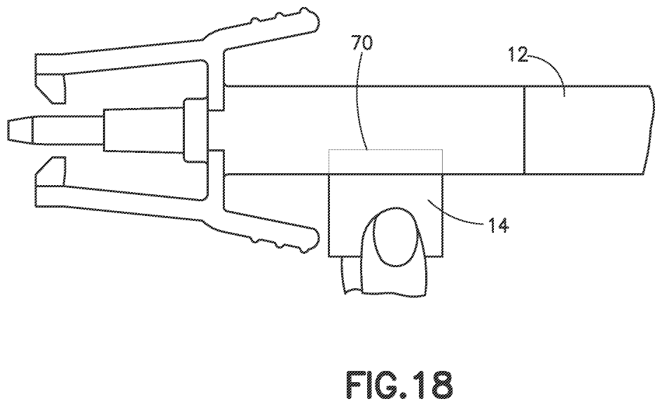

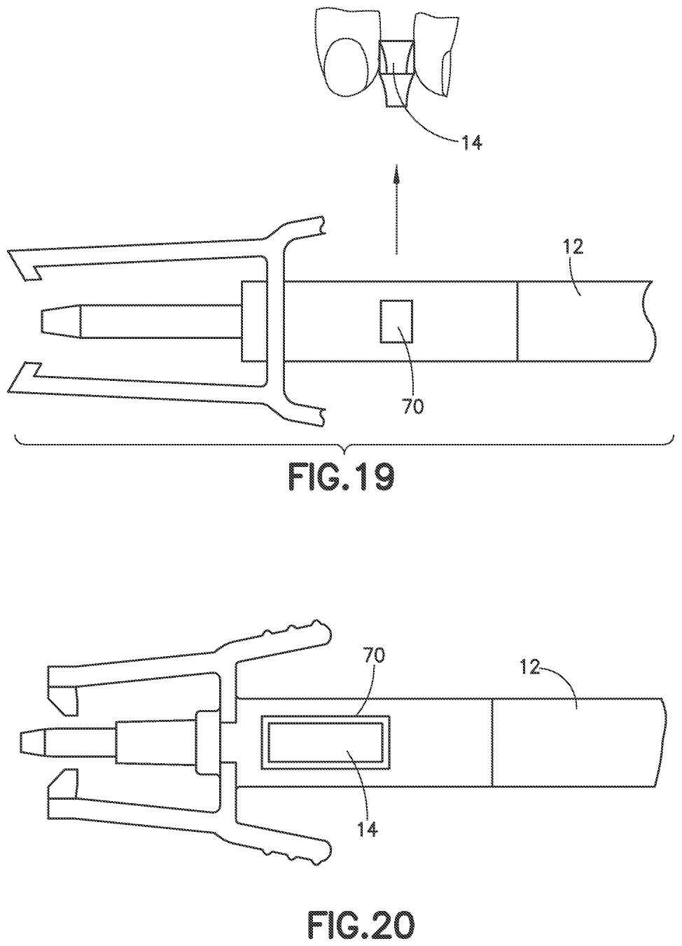

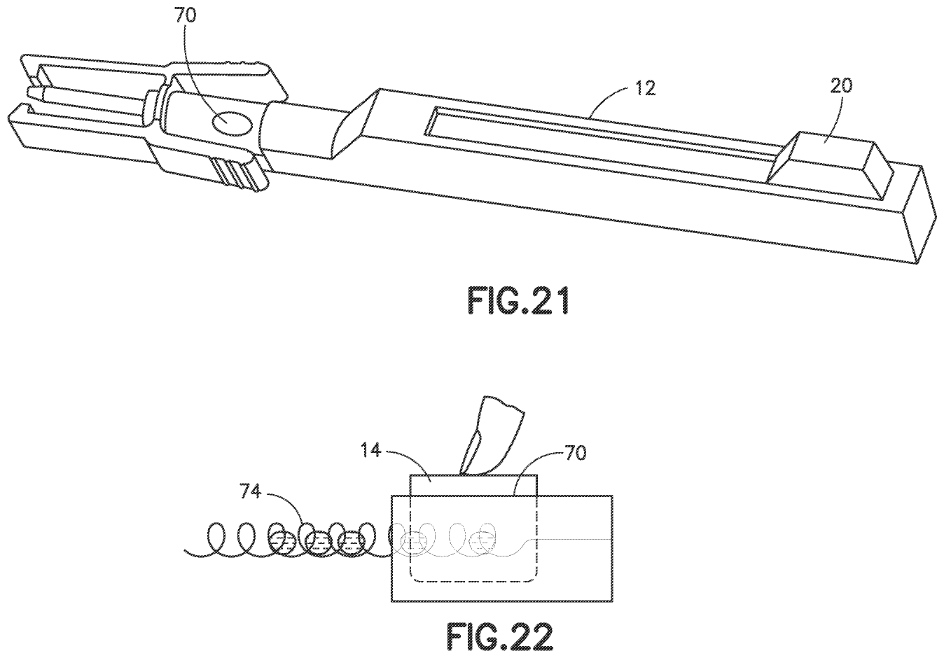

Referring to FIGS. 18-22, in a further aspect or embodiment, the housing 16 of the blood access device 12 includes a distal opening 70 configured to receive at least a portion of the diagnostic device 14. The diagnostic device 14 is placed in fluid communication with the blood access device 12 via the distal opening 70. As shown in FIG. 22, the instrument 18 may be a helical coil 74 that is configured to convey fluid from a patient's vasculature to the blood access device 12 and the diagnostic device 14.

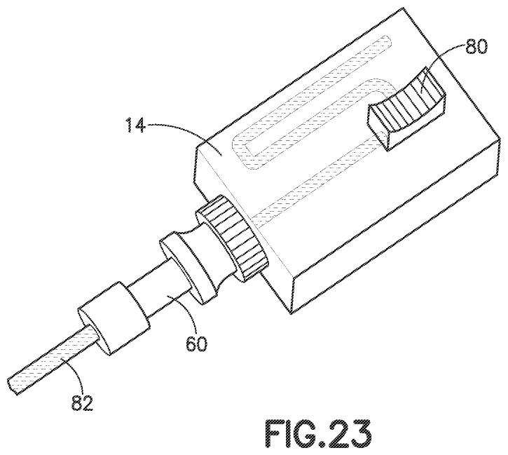

Referring to FIG. 23, in a further aspect or embodiment, the diagnostic device 14 includes an advancement element 80 connected to a tube 82, with the advancement element 80 configured to advance the tube 82 beyond the diagnostic device 14 in a similar manner as the instrument of the blood access device 12. The advancement element 80 and the tube 82 may be used in connection with the medical connector 60 described above, with the tube 82 configured to be pushed through a valve of the medical connector 60.

In one aspect or embodiment, the diagnostic device 14 is configured to receive a blood sample volume of 0.3 to 500 microliters. In one aspect or embodiment, the diagnostic device 14 is configured to receive a blood sample volume of 0.3 to 100 microliters.

Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the spirit and scope of the appended claims. For example, it is to be understood that the present invention contemplates that, to the extent possible, one or more features of any embodiment can be combined with one or more features of any other embodiment.

The invention claimed is:

1. A blood sample collection system comprising:
a blood access device having a fluid flow path; and
a diagnostic device configured to receive a sample of blood, wherein the diagnostic device is in fluid communication with the fluid flow path, and wherein the diagnostic device is configured to be detached from the blood access device.

2. The blood sample collection system of claim 1, wherein the blood access device comprises:
a housing,
an instrument disposed within the housing, and
an advancement element, wherein in response to movement of the advancement element with respect to the housing, the instrument is configured to advance beyond a distal end of the housing.

3. The blood sample collection system of claim 1, wherein the diagnostic device is configured to receive a sample volume of 0.3 to 500 microliters.

4. The blood sample collection system of claim 3, wherein the diagnostic device is configured to receive a sample volume of 0.3 to 100 microliters.

5. The blood sample collection system of claim 1, wherein the blood access device comprises a peripheral intravenous catheter.

6. The blood sample collection system of claim 1, wherein the blood access device comprises extension tubing.

7. The blood sample collection system of claim 1, wherein the diagnostic device comprises a diagnostic test cartridge.

8. The blood sample collection system of claim 7, wherein the blood access device comprises one of a cannula and septum, and wherein the diagnostic test cartridge comprises the other of the cannula and the septum, the cannula configured to extend through the septum to place the fluid flow path of the blood access device in fluid communication with the diagnostic test cartridge.

9. The blood sample collection system of claim 7, wherein a body of the diagnostic test cartridge is cylindrical.

10. The blood sample collection system of claim 7, wherein a body of the diagnostic test cartridge is planar.

11. The blood sample collection system of claim 2, wherein the advancement element comprises a cannula and the diagnostic device comprises a septum, the cannula configured to extend through the septum to place the fluid flow path of the blood access device in fluid communication with the diagnostic device.

12. The blood sample collection system of claim 2, wherein the housing of the blood access device comprises a distal opening configured to receive at least a portion of the diagnostic device.

13. The blood sample collection system of claim 2, wherein the instrument comprises a flow tube or a helical coil.

14. The blood sample collection system of claim 1, wherein the diagnostic device comprises a diagnostic test strip.

15. The blood sample collection system of claim 1, wherein the diagnostic device comprises a lateral flow assay.

16. The blood sample collection system of claim 1, wherein the diagnostic device comprises a visual indicator.

17. A blood sample collection system comprising:
a medical connector having a fluid flow path; and
a diagnostic device configured to receive a sample of blood, wherein the diagnostic device is in fluid communication with the fluid flow path, and wherein the diagnostic device is configured to be detached from the medical connector.

18. The blood sample collection system of claim 17, wherein the medical connector comprises one of a male luer connector and female luer connector, and wherein the diagnostic device comprises the other of the male luer connector and the female luer connector.

19. The blood sample collection system of claim 17, wherein the medical connector comprises a needle-free medical connector.

20. The blood sample collection system of claim 17, wherein the diagnostic device comprises a diagnostic test cartridge.
